(19) United States
(12) Patent Application Publication
(10) Pub. No.: US 2026/0357199 A1
(43) Pub. Date: Nov. 12, 2026

(54) MICROFLUIDIC DEVICES AND METHODS FOR THE DEVELOPMENT OF NEURAL TUBE-LIKE TISSUES OR NEURAL SPHEROIDS

(71) Applicant: The Regents of the University of Michigan, Ann Arbor, MI (US)

(72) Inventors: Xufeng Xue, Ann Arbor, MI (US); Jianping Fu, Ann Arbor, MI (US); Huaijing Tang, Ann Arbor, MI (US)

(21) Appl. No.: 19/736,387

(22) Filed: Jul. 9, 2026

Related U.S. Application Data
(62) Division of application No. 18/035,222, filed on Nov. 4, 2021.
(60) Provisional application No. 63/109,407, filed on Nov. 4, 2020.

(51) Int. Cl. C12N 5/079; C12M 1/12; C12M 3/00; C12M 3/06; C12N 5/00; C12N 5/071

(52) U.S. Cl. CPC C12N 5/0618; C12M 21/08; C12M 23/16; C12M 25/12; C12N 5/0068; C12N 5/0697; C12N 2513/00; C12N 2535/00

(57) ABSTRACT

The present disclosure provides devices and in vitro methods of developing three-dimensional neural tube-like tissues, forebrain spheroids, midbrain spheroids, hindbrain spheroids, and spinal cord spheroids. In some aspects, the disclosure provides devices and methods of developing three-dimensional neural tube-like tissues comprising forebrain-like, midbrain-like, hindbrain-like, and spinal cord-like tissues. In some aspects, the disclosure provides devices and methods of developing forebrain spheroids, midbrain spheroids, hindbrain spheroids, and spinal cord spheroids. In particular, provided herein microfluidic devices and methods of using the same for generating neural tube-like tissues, such as neural-tube like tissues comprising forebrain-like, midbrain-like, hindbrain-like, and spinal cord-like tissues. In particular, provided herein microfluidic devices and methods of using the same for generating forebrain spheroids, midbrain spheroids, hindbrain spheroids, and spinal cord spheroids. In some embodiments, uses of such neural tube-like tissues, forebrain spheroids, midbrain spheroids, hindbrain spheroids, and spinal cord spheroids for research, compound screening and analysis, disease modeling, and therapeutics are provided.

### STATEMENT OF RELATED APPLICATIONS

This application is a divisional of U.S. patent application Ser. No. 18/035,222, filed May 3, 2026, which is a national phase application under 35 U.S.C. § 371 of PCT International Application No.: PCT/US2021/058090, filed on Nov. 4, 2021, which claims priority to U.S. Provisional Patent Application No. 63/109,407, filed Nov. 4, 2020, the entire contents of which are incorporated herein by reference for all purposes.

### STATEMENT OF FEDERAL FUNDING

This invention was made with government support under NS113518 awarded by the National Institutes of Health, and CBET1901718 awarded by the National Science Foundation. The government has certain rights in the invention.

### FIELD

The present disclosure provides systems, devices, and methods of developing three-dimensional neural tube-like tissues and/or neural spheroids in vitro. In particular, provided herein microfluidic devices and methods of using the same for generating neural tube-like tissues or neural spheroids. In some embodiments, uses of such neural tube-like tissues or neural spheroids for research, compound screening and analysis, and therapeutics are provided.

### BACKGROUND

The neural tube is the embryonic progenitor to the central nervous system. The posterior region of the neural tube gives rise to the spinal cord, whereas the anterior region becomes the brain (containing forebrain, midbrain and hindbrain). An important process during the development of the neural tube is the cell fate specification and patterning along the dorsal-ventral and anterior-posterior axes, which leads to the differentiation of distinct classes of neuronal progenitor cells located at defined positions within the neural tube. These neuronal progenitor cells ultimately give rise to distinct classes of mature functional neurons.

In recent years, tremendous progress has been achieved in understanding the molecular mechanisms of neuronal cell fate specification along the dorsal-ventral and anterior-posterior axes in the neural tube. However, the details of regional neuronal cell fate specification, such as the interaction between signaling pathways, remain largely unknown. Moreover, current in vitro methods for developing neural tissues fail to produce a neural tube-like tissue architecture with proper dorsal-ventral and anterior-posterior patterning mimicking that seen in the human neural tube.

Accordingly, devices and methods for generating human neural tube-like tissues with effective anterior-posterior and dorsal-ventral patterning are needed. The development of such devices and methods, and human neural tube-like tissues generated using the same, may benefit studies of human neurodevelopment and provide an advanced technological platform for screening of potential therapeutic compounds and the effects of chemicals and drugs on neurodevelopmental toxicity.

### SUMMARY

Provided herein are devices, systems, and methods for generating three-dimensional neural tube-like tissues or neural spheroids. Further provided herein are methods for using neural tube-like tissues or neural spheroids for various applications.

In some aspects, provided herein are microfluidic devices for generating three-dimensional neural tube-like tissues or neural spheroids. The device comprises a central channel, a top channel, and a bottom channel. The top channel is parallel to the central channel and is separated from the central channel by a first semi-permeable structure. The bottom channel is parallel to the central channel, and is separated from the central channel by a second semi-permeable structure. The first semi-permeable structure and the second semi-permeable structure may comprise a plurality of circular microposts.

In some embodiments, the central channel comprises at least one reservoir in fluid connection with the central channel. For example, the central channel may comprise a right reservoir and a left reservoir, wherein each reservoir is in fluid connection with the central channel.

In some embodiments, the top channel comprises at least one reservoir in fluid connection with the top channel, and the bottom channel comprises at least one reservoir in fluid connection with the bottom channel. For example, the top channel may comprise a right reservoir and a left reservoir, wherein each reservoir is in fluid connection with the top channel; and the bottom channel may comprise a right reservoir and a left reservoir, wherein each reservoir is in fluid connection with the bottom channel.

The central channel comprises a plurality of cell-attachment islands positioned on a bottom surface of the central channel. In some embodiments, the cell-attachment islands comprise an adhesive protein matrix. In some embodiments, the cell-attachment islands are rectangular in shape. In some embodiments, the cell-attachment islands are circular in shape. In some embodiments, at least 4 cell-attachment islands are positioned on the bottom surface of the central channel. The cell-attachment islands may be aligned parallel to each other on the bottom surface of the central channel. In some embodiments, the cell-attachment islands are formed by microcontact printing onto a coverslip. The coverslip may comprise glass. In some embodiments, the coverslip is a base for the microfluidic device, such that the coverslip is a bottom surface for each of the top channel, central channel, and bottom channel.

In some aspects, provided herein are systems comprising a device as described herein and cells contained therein. For example, provided herein are systems comprising a microfluidic device as described herein and cells contained in the central channel thereof. The cells may comprise stem cells. The system may further comprise one or more chemicals. For example, the system may comprise a suitable combination of chemicals used in the compositions described herein to induce anterior-posterior and/or dorsal-ventral patterning of the stem cells contained within the central channel. For example, the system may comprise a TGF-β inhibitor, a bone morphogenic protein 4 (BMP4) inhibitor, a WNT activator, fibroblast growth factor 8 (FGF8), BMP4, retinoic acid, sonic hedgehog (SHH), and smoothened agonist (SAG). In some embodiments, the stem cells contained within the system may be differentiated into a three-dimensional neural tube-like tissue exhibiting anterior-posterior and dorsal-ventral patterns mimicking those seen in the human neural tube. In some embodiments, the stem cells contained within the system may be differentiated into spherical three-dimensional neural tissues (e.g. neural spheroids) mimicking human forebrain, midbrain, hindbrain and spinal cord tissues in the neural tube. For example, the stem cells may be differentiated into one or more forebrain spheroids, midbrain spheroids, hindbrain spheroids, and/or spinal cord spheroids.

In some aspects, provided herein are methods for generating a neural tube-like tissue. The methods comprise providing a microfluidic device as described herein and introducing stem cells into the central channel. The methods further comprise injecting a gel matrix into the central channel. The methods further comprise producing a first chemical gradient along the length of the central channel, and producing an orthogonal chemical gradient along the radius of the central channel.

In some aspects, provided herein are methods for generating neural spheroids. For example, provided herein are methods for generating one or more forebrain spheroids, midbrain spheroids, hindbrain spheroids, or spinal cord spheroids. The methods comprise providing a microfluidic device as described herein and introducing stem cells into the central channel. The methods further comprise injecting a gel matrix into the central channel. The methods further comprise producing a first chemical gradient along the length of the central channel, and producing an orthogonal chemical gradient along the radius of the central channel.

In some embodiments, the methods comprise providing a microfluidic device as described herein, introducing stem cells into the central channel, and promoting attachment of the stem cells to the cell-attachment islands. In some embodiments, the cell-attachment islands are rectangular in shape. In some embodiments, the cell-attachment islands are circular in shape. In some embodiments, the stem cells comprise human pluripotent stem cells (hPSCs), including both human embryonic stem cells (hESCs) and human induced pluripotent stem cells (hiPSCs). Promoting attachment to the cell-attachment islands may comprise incubating the stem cells within the central channel for 16 hours at 37° C. The methods further comprise injecting a gel matrix into the central channel and promoting formation of a tubular structure or a spherical structure. In some embodiments, promoting formation of a tubular structure or a spherical structure comprises incubating the stem cells and the gel matrix within the central channel for 24 hours at 37° C.

The methods further comprise producing a first chemical gradient along the length of the central channel, and producing an orthogonal chemical gradient along the radius axis of the central channel. In some embodiments, production of the first chemical gradient and the orthogonal chemical gradient promotes generation of a neural tube-like tissue exhibiting anterior-posterior and dorsal-ventral patterns mimicking those seen in the human neural tube. In some embodiments, production of the first chemical gradient and the orthogonal chemical gradient promotes generation of human forebrain spheroids, midbrain spheroids, hindbrain spheroids, and/or spinal cord spheroids. In some embodiments, the first chemical gradient and the orthogonal chemical gradient are produced simultaneously. In some embodiments, the first chemical gradient is produced by applying a first composition to the left reservoir of the central channel and applying a second composition to the right reservoir of the central channel. In some embodiments, the orthogonal chemical gradient is produced by applying a third composition to the top channel and applying a fourth composition to the bottom channel. In some embodiments, the third composition is applied to the left and right reservoirs of the top channel and the fourth composition is applied to the left and right reservoirs of the bottom channel.

In some aspects, provided herein are compositions comprising a neural tube-like tissue generated using the devices, systems, and methods described herein. In some aspects, provided herein are compositions comprising neural spheroids generated using the devices, systems, and methods described herein. Such compositions find use in methods of testing compounds, such as for therapeutic applications or for toxicity screening.

In some aspects, provided herein are methods for testing one or more compounds. The methods comprise providing a composition as described herein, exposing a test compound to the composition, and determining an effect of the test compound on the composition. For example, the effect may be the presence or absence of toxicity.

### DESCRIPTION OF THE FIGURES

The patent or application file contains at least one drawing executed in color. Copies of this patent or patent application publication with color drawings will be provided by the Office upon request and payment of the necessary fee.

FIG. 1A-1C is a schematic showing microcontact printing, device assembly, cell loading, gel loading and anterior-posterior patterning of the human neural tube-like tissue. (FIG. 1A) Rectangular Geltrex islands were printed onto a coverslip using microcontact printing with a polydimethylsiloxane (PDMS) stamp. (FIG. 1B) A PDMS structural layer was attached onto the coverslip with Geltrex islands aligned in the center of the central channel. Dissociated single human pluripotent stem cells (hPSCs) were loaded into the central channel and attached onto the Geltrex islands to form rectangular cell colonies. (FIG. 1C) hPSCs formed an elongated tubular structure with a central lumen after Geltrex was introduced into the central channel. Both the left and right medium reservoirs of the central channel were filled with the neural induction medium. In the right medium reservoir of the central channel, the WNT activator CHIR99021 (CHIR), fibroblast growth factor 8 (FGF8) and retinoic acid (RA) were further supplemented into the neural induction medium to establish a chemical gradient along the central channel to induce anterior-posterior patterning of the human neural tube-like tissue. Here, the growth medium comprised DMEM/F12 (GIBCO), 20% KnockOut Serum Replacement (KSR; GIBCO), β-mercaptoethanol (0.1 mM; GIBCO), glutamax (2 mM; GIBCO), 1% non-essential amino acids (GIBCO), and human recombinant basic fibroblast growth factor (bFGF, 4 ng ml⁻¹; GlobalStem). The neural induction medium (DSi in the figure) comprised the growth medium, TGF-β inhibitor SB 431542 (10 μM; Cayman Chemical) and BMP4 inhibitor LDN 193189 (500 nM; Selleckchem).

FIG. 2A-2E show anterior-posterior patterned, human neural tube-like tissues with CHIR, FGF8 and RA supplemented into the right medium reservoir of the central channel. (FIG. 2A) Protocol for generating anterior-posterior patterned human neural tube-like tissues with CHIR, FGF8 and RA supplemented into the right medium reservoir of the central channel. hPSCs were seeded into the central channel on day 0. After gel loading into the central channel on day 1, hPSCs started to form 3D tubular structures with a single central lumen. From day 2 to day 5, CHIR, FGF8 and RA added into the right medium reservoir of the central channel induced the posteriorization and thus anterior-posterior patterning of the neural tube-like tissue. All tissues were analyzed on day 4 or day 7. (FIG. 2B-FIG. 2D) Stitched confocal micrographs showing patterned gene expression of the human neural tube-like tissues on day 4 or day 7. The human neural tube-like tissues were stained for (FIG. 2B) HOXB1, HOXB4 and HOXC9 at day 7; (FIG. 2C) OTX2, HOXB1 and HOXB4 at day 7; (FIG. 2D) CDX2, SOX2 and TBXT at day 4. OTX2, HOXB1, HOXB4 and HOXC9 were expressed sequentially from the anterior end to the posterior end of the human neural tube-like tissues at day 7. CDX2 and TBXT were expressed at the posterior end of the neural tube-like tissue at day 4. In B & D, zoom-in views of the indicated boxed regions were shown. In B & C, stitched confocal micrographs showing an array of neural tube-like tissues at day 7 are presented. (FIG. 2E) The human neural tube-like tissue was physically dissected using surgical knives into four even segments (region 1-4) for downstream qRT-PCR analysis. Heatmaps showed normalized expression of forebrain (OTX2), midbrain (PAX8), and hindbrain (GBX2) markers, signaling molecules (FGF8, CYP26A1, CYP26B1 and ALDH1A2), and HOX genes in the four regions of the anterior-posterior patterned neural tube-like tissue.

FIG. 3A-3D show anterior-posterior and dorsal-ventral patterned human neural tube-like tissues. (FIG. 3A) Protocol for generating anterior-posterior and dorsal-ventral patterned human neural tube-like tissue. From day 2 to day 5, CHIR, FGF8 and RA were added into the right medium reservoir of the central channel to induce anterior-posterior patterning of the neural tube-like tissue. From day 5 to day 7, BMP and RA/smoothened agonist (SAG) were added into the top and bottom channels, respectively, to induce dorsal-ventral patterning of the neural tube-like tissue. (FIG. 3B-3D) Stitched confocal micrographs showing anterior-posterior and dorsal-ventral patterned neural tube-like tissues on day 7. In FIG. 3B, the neural tube-like tissues were stained for HOXB1, HOXB4, and HOXC9. In FIG. 3C, the neural tube-like tissues were stained for OTX2 (forebrain and midbrain marker) and HOXB1. In FIG. 3D, the neural tube-like tissues were stained for PAX3 (dorsal neural tube marker) and OLIG2 (motor neuron progenitor marker). Dorsal neural tube marker PAX3 was expressed on the dorsal side of the neural tube-like tissues, while ventral neural tube marker OLIG2 was expressed on the opposite ventral side of the neural tube-like tissue. Zoom-in views are provided for the boxed regions as indicated.

FIG. 4A-4C show cross-sectional images of the forebrain region of anterior-posterior and dorsal-ventral patterned human neural tube-like tissues. These human neural tube-like tissues were generated using the protocol in FIG. 3A, with the culture time extended to Day 21. (FIG. 4A) Cartoon showing the location where cross-sectional images were taken for anterior-posterior and dorsal-ventral patterned human neural tube-like tissue at day 21. (FIG. 4B) Confocal micrographs showing cross-sections of the forebrain region of the patterned human neural tube-like tissue at day 21, stained for PAX6, DLX5, NKX2.1, OTX2, SOX2, MAP2, and ZO-1. PAX6 is a dorsal forebrain marker, whereas DLX5 and NKX2.1 are ventral forebrain markers. (FIG. 4C) Zoom-in views for PAX6, DLX5, and NKX2.1 staining, for the boxed regions selected in FIG. 4B as indicated.

FIG. 5A-5B show cross-sectional images of the spinal cord region of anterior-posterior and dorsal-ventral patterned human neural tube-like tissues. These human neural tube-like tissues were generated using the protocol in FIG. 3A, with the culture time extended to Day 21. (FIG. 5A) Cartoon showing the location where cross-sectional images were taken for anterior-posterior and dorsal-ventral patterned human neural tube-like tissue at day 21. (FIG. 5B) Confocal micrographs showing cross-sections of the spinal cord region of the patterned human neural tube-like tissue at day 21, stained for PAX3, OLIG2, SOX10, and NKX6.1. PAX3: a dorsal neural tube marker; SOX10: a neural crest marker; OLIG2 and NKX6.1: ventral spinal cord markers. Zoom-in views for the boxed regions are provided as indicated.

FIG. 6A-6C show the development of forebrain-like, midbrain-like, hindbrain-like, and spinal cord-like tissues (e.g. spheroids) from patterned circular human pluripotent stem cell colonies in the microfluidic device. These tissues were generated using the protocol in FIG. 2A. (FIG. 6A) Protocol for generating forebrain spheroids, midbrain spheroids, hindbrain spheroids, and spinal cord spheroids with CHIR, FGF8 and RA supplemented into the right medium reservoir of the central channel. Human pluripotent stem cells were seeded into the central channel on day 0 to form a regular array of circular cell colonies. After gel loading into the central channel on day 1, human pluripotent stem cells started to form 3D spherical structures with a single central lumen. From day 2 to day 5, CHIR, FGF8 and RA were added into the right medium reservoir of the central channel. This induced posteriorization and thus anterior-posterior patterning of these tissues to form forebrain-, midbrain-, hindbrain-and spinal cord-like tissues (e.g. spheroids) as indicated. All tissues were analyzed on day 7. (B) Bright field image showing a regular array of circular cell colonies on day 7 in the central channel. (C) Confocal micrographs showing forebrain spheroids, midbrain spheroids, hindbrain spheroids, and spinal cord spheroids at day 7, stained for PAX6, OTX2, HOXB1, HOXB4, and HOXC9 as indicated. PAX6: forebrain marker; OTX2: forebrain and midbrain marker; HOXB1: hindbrain marker; HOXB4 and HOXC9: spinal cord marker.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the term “microcontact printing” refers to a form of soft lithography that uses relief patterns on a master stamp to form patterns on a surface of a substrate through conformal contact.

As used herein, the term “neural tube” refers to the embryonic precursor to the central nervous system (e.g. the brain and the spinal cord). The neural tube patterns along the dorsal-ventral and anterior-posterior axes to establish defined compartments of neural progenitor cells that lead to distinct classes of neurons.

As used herein, the term “neural tube-like tissue” refers to tissues differentiated in vitro (e.g., from a stem cell) that has one or more properties of the neural tube (e.g. dorsal-ventral patterning, anterior-posterior patterning).

As used herein, the term “neural spheroid” refers to a tissue differentiated in vitro (e.g., from a stem cell) that has one or more properties of a tissue or portion thereof in the mature or developing nervous system. A neural spheroid is roughly spherical in shape. For example, a neural spheroid may comprise forebrain-like, midbrain-like, hindbrain-like, and/or spinal cord-like spherical tissues. A forebrain-like spherical tissue, referred to herein as a “forebrain spheroid”, comprises one or more properties of forebrain tissues in the neural tube. For example, a forebrain spheroid may comprise a gene expression pattern, specific cell type (e.g. neuronal cell type), and/or cell organization similar to that seen in the forebrain tissues in the neural tube. A midbrain-like spherical tissue, referred to herein as a “midbrain spheroid”, comprises one or more properties of the midbrain tissues in the neural tube. For example, a midbrain spheroid may comprise a gene expression pattern, specific cell type (e.g. neuronal cell type), and/or cell organization similar to that seen in the midbrain tissues in the neural tube. A hindbrain-like spherical tissue, referred to herein as a “hindbrain spheroid”, comprises one or more properties of the hindbrain tissues in the neural tube. For example, a hindbrain-like tissue may comprise a gene expression pattern, specific cell type (e.g. neuronal cell type), and/or cell organization similar to that seen in the hindbrain tissues in the neural tube. A spinal cord-like spherical tissue, referred to herein as a “spinal cord spheroid”, comprises one or more properties of the spinal cord tissues in the neural tube. For example, a spinal cord spheroid may comprise a gene expression pattern, specific cell type (e.g. neuronal cell type), and/or cell organization similar to that seen in the spinal cord tissues in the neural tube.

The term “administration” and variants thereof (e.g., “administering” a compound) in reference to cells or a compound means providing the cells or compound or a prodrug of the compound to the individual in need of treatment or prophylaxis. When cells or a compound of the technology or a prodrug thereof is provided in combination with one or more other active agents, “administration” and its variants are each understood to include provision of the compound or prodrug and other agents at the same time or at different times. When the agents of a combination are administered at the same time, they can be administered together in a single composition or they can be administered separately. As used herein, the term “composition” is intended to encompass a product comprising the specified ingredients in the specified amounts, as well as any product that results, directly or indirectly, from combining the specified ingredients in the specified amounts.

By “pharmaceutically acceptable” is meant that the ingredients of the pharmaceutical composition are compatible with each other and not deleterious to the recipient thereof.

The term “subject” as used herein refers to an animal, preferably a mammal, most preferably a human, who has been the object of treatment, observation, or experiment.

The term “effective amount” as used herein means that amount of an agent (e.g., amnion-like tissue) that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented.

### DETAILED DESCRIPTION

The foundation for the anatomical and functional complexity of the vertebrate central nervous system (CNS) is laid during the development of neural tube, the embryonic precursor to the CNS. Development of the neural tube starts from the formation of neural plate in dorsal ectoderm through the neural induction process, before its infolding into a tubular structure enclosing a central fluid-filled lumen (the neural canal). Continuous development of the neural tube involves patterning and differentiation of distinct classes of neuronal progenitor cells located at defined positions within the neural tube along the anterior-posterior and dorsal-ventral axes. Neuronal patterning along the anterior-posterior axis establishes the main subdivisions of CNS: forebrain, midbrain, hindbrain and spinal cord. The development of human neural tube is a tightly regulated, genetically encoded process. Any deviation from the normal neural tube developmental program can result in neural tube defects and neurodevelopmental disorders and may lead to distinct neurological and psychiatric diseases later in life.

An important process during early neurodevelopment is regional neuronal cell fate specification and patterning along the dorsal-ventral and anterior-posterior axes, which leads to the differentiation of distinct classes of neuronal progenitor cells located at defined positions within the neural tube. These neuronal progenitor cells ultimately give rise to distinct classes of mature neurons. In recent years, tremendous progress has been achieved in understanding the molecular mechanisms of dorsal-ventral and anterior-posterior patterning of the neural tube. Neuronal fate within the neural tube is directed by factors that are secreted from local surrounding tissues. Thus, the position of neural progenitor cells in the neural tube influences their ultimate fate by defining the identity and concentration of inductive signals to which they are exposed.

Current models of neural tube patterning indicate that a few predominant signaling molecules jointly coordinate most of the spatial and temporal differentiation of the neural tube. These molecules are sonic hedgehog (SHH), bone morphogenic protein (BMP), retinoic acid (RA), fibroblast growth factor (FGF), and WNT molecules. SHH is generated in the notochord and floor plate cells establishing a ventral to dorsal concentration gradient across the neural tube. An opposing gradient of BMPs and other members of the transforming growth factor beta (TGFβ) superfamily of signaling molecules are simultaneously released by roof plate cells, thus concurrently patterning the dorsal portion of the neural tube. Patterning along the anterior-posterior axis occurs simultaneously with dorsal-ventral patterning and is thought to be the result of opposing gradients of RA and FGF/WNT.

However, the details of regional neuronal cell fate specification and patterning along the dorsal-ventral and anterior-posterior axes of the neural tube, such as the interaction between signaling pathways, remain largely unknown. Accordingly, devices and methods for generating neural tube-like tissues, in particular neural tube-like tissues containing region-specific human neural tissues (such as forebrain-like, midbrain-like, hindbrain-like, and spinal cord-like tissues) may benefit studies of the neural tube development. Moreover, devices and methods for generating neural tube-like tissues with dorsal-ventral and anterior-posterior patterning effectively mimicking that seen in the human neural tube may enable effective in vitro models to study the neural tube development and disorders thereof, and provide an advanced platform for screening of potential therapeutic compounds and the impacts of chemicals and drugs on neurodevelopmental toxicity.

### I. Devices for Generating Tissues

In some aspects, provided herein are devices for generating tissues. In some aspects, provided herein are devices for generating neural tube-like tissues. In some aspects, provided herein are devices for generating neural spheroids. For example, in some embodiments provided herein are devices for generating neural spheroids, such as forebrain spheroids, midbrain spheroids, hindbrain spheroids, and/or spinal cord spheroids.

In some embodiments, the devices are microfluidic devices. In some embodiments, the device comprises a central channel. In some embodiments, the device further comprises a top channel and a bottom channel. In some embodiments, the channels are arranged on a planar surface. In some embodiments, the top channel is separated from the central channel by a first semi-permeable structure and the bottom channel is separated from the central channel by a second semi-permeable structure. The first and second semi-permeable structure may comprise the same material or different materials. In some embodiments, the first and second semi-permeable structures comprise a plurality of microposts (e.g. circular microposts). The microposts may be arranged in a suitable manner to permit exchange of chemicals between the bottom channel and the central channel and between the top channel and the central channel, while prohibiting the movement of a gel matrix away from the central channel (e.g. prohibiting movement of the gel matrix from the central channel to the top or bottom channels). The desired exchange of chemicals may be permitted by suitable spacing of the microposts from one another.

In some embodiments, the central channel comprises at least one reservoir in fluid connection with the channel. In some embodiments, the central channel comprises a right reservoir and a left reservoir, wherein each reservoir is in fluid connection with the central channel. Accordingly, chemicals may be applied to the reservoir(s) of the central channel to influence the development of tissues (e.g. neural tube-like tissues, neural spheroids) within the central channel. For example, chemical gradients may be established in the central channel, by applying different chemicals with different concentrations to the reservoir(s) of the central channel, to influence the anterior-posterior patterning of a neural tube-like tissue or of a neural spheroid within the central channel.

In some embodiments, the top channel and the bottom channel each comprise at least one reservoir in fluid connection with the channel. In some embodiments, the top channel comprises a right reservoir and a left reservoir, wherein each reservoir is in fluid connection with the top channel. In some embodiments, the bottom channel comprises a right reservoir and a left reservoir, wherein each reservoir is in fluid connection with the bottom channel. Accordingly, different chemicals with different concentrations may be applied to the reservoir(s) of the top channel and/or of the bottom channel. The chemicals may flow from the reservoirs to the main body of the respective channel, and subsequently may contact and/or influence the developing tissue (e.g. neural tube-like tissue, neural spheroid) through the semi-permeable membrane connecting the channel (e.g. top channel or bottom channel) with the central channel.

In some embodiments, the central channel comprises at least one sample introduction site (e.g. injection site). For example, the central channel may comprise an injection site allowing for injection of stem cells, culture medium, gel matrix, or other desired components into the central channel. In some embodiments, the central channel comprises a single injection site used for introduction/injection of stem cells, culture medium, and gel matrix to the central channel.

In some embodiments, the central channel comprises a plurality of cell-attachment islands. In some embodiments, the plurality of cell-attachment islands are positioned on a bottom surface of the central channel. Any suitable number of cell-attachment islands may be positioned on the bottom surface of the central channel. In some embodiments, the device comprises at least 2, at least 3, at least 4, at least 5, at least 6, at least 7, at least 8, at least 9, or more than 10 cell attachment islands. The cell attachment islands may be of any suitable shape and size and of any suitable spacing from one another. In some embodiments, the cell attachment islands are regular shapes. For example, the cell attachment may be a square, a circle, an equilateral triangle, a pentagon, a hexagon, a heptagon, an octagon, etc. In some embodiments, the cell-attachment islands are longer in a first dimension (e.g. length) than a second dimension (e.g. width). In some embodiments, the cell-attachment islands are rectangular in shape. For example, the cell-attachment islands may comprise a plurality of rectangles (e.g. at least 2, at least 3, at least 4, at least 5, at least 6, at least 7, at least 8, at least 9, at least 10) that are arranged parallel to each other on the bottom surface of the central channel. In some embodiments, cell-attachment islands that are arranged parallel to each other on the bottom surface of the central channel may be particularly useful for generating neural-tube like structures. In some embodiments, the cell-attachment islands are circular in shape. For example, the cell-attachment islands may comprise a plurality of circles (e.g. at least 2, at least 3, at least 4, at least 5, at least 6, at least 7, at least 8, at least 9, at least 10). In some embodiments, the plurality of cell-attachment islands (e.g. plurality of circular cell-attachment islands) are arranged in regular arrays on the bottom surface of the central channel. Circular cell-attachment islands arranged in a regular array may be particularly useful for generating neural spheroids.

The cell-attachment islands comprise a suitable material to promote attachment of cells to the cell-attachment islands. In some embodiments, the cell-attachment islands comprise a suitable material to promote attachment of stem cells to the islands. In some embodiments, the cell-attachment islands comprise a gel matrix. The present disclosure is not limited to particular gel matrices. In some embodiments, the gel matrix is a natural or synthetic polymeric hydrogel (e.g., polyethylene glycol (PEG) hydrogels, poly (2-hydroxyethyl methacrylate) (PHEMA) hydrogels, growth factor basement membrane matrix (e.g. Geltrex), gelatinous protein mixture secreted by Engelbreth-Holm-Swarm (EHS) mouse sarcoma cells (Matrigel hydrogel), collagen, vitronectin, hyaluronic acid (HA), fibrin, or a combination thereof). In some embodiments, commercially available matrices (e.g., available from Fisher Scientific (Waltham, MA), Amsbio (Abingdon, UK), Corning (Corning, NY), or Trevigen, Inc. (Gaithersburg, MD)) are utilized. In some embodiments, the cell-attachment islands comprise a diluted gel matrix. For example, the cell-attachment islands may comprise a gel matrix (e.g. Geltrex) that is diluted with a suitable medium to 10%, 20%, 30%, 40%, 50%, 60%, 70%, 80%, or 90% of the original concentration of the gel matrix.

In some embodiments, the cell-attachment islands are formed by microcontact printing onto the bottom surface of the central channel. For example, the cell-attachment islands may be formed by microcontact printing onto a coverslip. Generally speaking, microcontact printing is performed using a PDMS stamp. However, other suitable polymers may be used for the stamp (e.g. polydimethylsiloxanes, polyurethanes, polyimides, novalac resins, etc.). The stamp (e.g. PDMS stamp) may be coated with the desired material for the cell-attachment islands (e.g. diluted gel matrix), and subsequently contacted with the coverslip. Conformal contact between the stamp and the coverslip results in transfer of the material for the cell-attachment islands (e.g. diluted gel matrix) onto the surface of the coverslip in the desired arrangement (e.g. parallel rectangular cell-attachment islands or arrays of circular cell-attachment islands). The coverslip may comprise any suitable material to promote adherence of the cell-attachment islands to the coverslip. In some embodiments, the coverslip may be a glass coverslip.

In some embodiments, the coverslip serves as the bottom surface for the entire microfluidic device. Accordingly, the coverslip may be the bottom surface for each of the central channel, the top channel, and the bottom channel, including all reservoirs (e.g. left reservoirs and right reservoirs) therefor.

### II. Methods for Generating Tissues

In some aspects, provided herein are methods for generating tissues. In some embodiments, provided herein are methods for generating neural tube-like tissues. In some embodiments, provided herein are methods for generating neural spheroids. For example, in some embodiments provided herein are methods for generating forebrain spheroids, midbrain spheroids, hindbrain spheroids, and/or spinal cord spheroids.

The methods are performed using a microfluidic device as described herein. The methods comprise introducing (e.g. injecting) stem cells into the central channel containing the cell attachment islands. In some embodiments, the stem cells are injected into the central channel through an injection site on the device. For example, the device may comprise an injection site allowing the introduction of stem cells into the central channel and subsequent adherence (e.g. seeding) of the stem cells onto the cell-attachment islands.

The stem cells may reside within the central channel in the presence of a suitable cell culture medium. The medium may be selected based upon the type of stem cell used. In some embodiments, the cell culture medium may be a suitable medium for the maintenance and/or expansion of human pluripotent stem cells, including human embryonic stem cells or human induced pluripotent stem cells. In some embodiments, the stem cells are injected into the central channel simultaneously with the culture medium (e.g. the stem cells are within the culture medium, and the medium containing the stem cells is injected into the central channel.). In some embodiments, the medium is added to the central channel prior to injection of the stem cells.

The stem cells may be any suitable stem cells, including pluripotent stem cells (embryonic stem cells and induced pluripotent stem cells or iPSCs), totipotent stem cells, expanded potential stem cells, and neural stem cells, regardless of source or species. For example, induced pluripotent stem cells may be derived from stem cells or adult somatic cells that have undergone a dedifferentiation process. Pluripotent stem cells, totipotent stem cells and neural stem cells may be human cells or be associated with other species (for example, monkey, pig and cow). In particular embodiments, the stem cells are human stem cells.

Induced pluripotent stem cells may be generated using any known approach. In some embodiments, iPSCs are obtained from adult human cells (e.g., fibroblasts). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, 1-myc), Nanog, LIN28, Glis1, etc.) or mimicking their activities is employed to generate iPSCs (using transgenic vectors (adenovirus, lentivirus, plasmids, transposons, etc.), inhibitors, delivery of proteins, microRNAs, etc.).

Totipotent stem cells may be generated using any known approach. In some embodiments, totipotent stem cells are obtained from pluripotent stem cells (for example, embryonic stem cells). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, 1-myc), Nanog, LIN28, Glis1, etc.) or mimicking their activities is employed to generate totipotent stem cells (using transgenic vectors (adenovirus, lentivirus, plasmids, transposons, etc.), inhibitors, delivery of proteins, microRNAs, etc.).

In some embodiments, the cells are expanded potential stem cells (Yang et al., Nature volume 550, pages 393-397(2017); herein incorporated by reference in its entirety), trophoblast stem cells (Okae, et al., Cell Stem Cell. 2018 Jan. 4; 22(1): 50-63.e6; herein incorporated by reference in its entirety), or hypoblast stem cells (Linneberg-Agerholm, et al. Development. 2019 Dec. 16; 146(24):dev180620; herein incorporated by reference in its entirety). In some embodiments the cells are not human cells and are associated with other mammalian species (for example, monkey, pig, or cow). In some embodiments the cells are non-terminally differentiated cells (regardless of pluripotency) or other non-maturated cells. In some embodiments, cells are screened for propensity to develop teratomas or other tumors (e.g., by identifying genetic lesions associated with a neoplastic potential). Such cells, if identified and undesired, are discarded.

The methods further comprise injecting a gel matrix into the central channel. In some embodiments, the gel matrix is injected into the central channel following injection of the stem cells. For example, the stem cells may be injected into the central channel and incubated in the presence of a suitable cell culture medium for a suitable duration of time to allow seeding of the stem cells onto the cell-attachment islands. In some embodiments, the stem cells are incubated in the cell culture medium for 10-24 hours at 37° C. In some embodiments, the stem cells are incubated in the presence of a suitable cell culture medium within the central channel for 16 hours at 37 ° C. to promote seeding of the stem cells to the cell-attachment islands. Following suitable adherence of the stem cells to the cell-attachment islands, the gel matrix may be injected into the central channel. Any suitable gel matrix may be employed. In some embodiments, the gel matrix is a natural or synthetic polymeric hydrogel (e.g., polyethylene glycol (PEG) hydrogels, poly(2-hydroxyethyl methacrylate) (PHEMA) hydrogels, growth factor basement membrane matrix (e.g. Geltrex), gelatinous protein mixture secreted by Engelbreth-Holm-Swarm (EHS) mouse sarcoma cells (Matrigel hydrogel), collagen, vitronectin, hyaluronic acid (HA), fibrin, or a combination thereof). In some embodiments, commercially available matrices (e.g., available from Fisher Scientific (Waltham, MA), Amsbio (Abingdon, UK), Corning (Corning, NY), or Trevigen, Inc. (Gaithersburg, MD) are utilized.

The gel matrix provides a three-dimensional environment in which cells will spontaneously form a three-dimensional structure (either tubular structure or spherical structure) with a central apical lumen. In some embodiments, cells spontaneously form a three-dimensional tubular structure with a central apical lumen. For example, devices comprising a plurality of rectangular cell-attachment sites arranged in parallel to each other may be particularly useful for allowing spontaneous generation of a tubular structure. In some embodiments, cells spontaneously form a spherical structure with a central apical lumen. For example, devices comprising a plurality of circular cell attachment sites arranged in an array may be particularly useful for allowing spontaneous generation of a spherical structure (e.g. a neural spheroid). Following the formation of the three-dimensional structure, methods for inducing anterior-posterior and dorsal-ventral patterning of the three-dimensional structure may be performed.

In some embodiments, the methods further comprise producing a first chemical gradient along the length of the central channel. In some embodiments, producing the first chemical gradient along the length of the channel allows for the generation of a neural tube-like tissue with anterior-posterior patterning mimicking those seen in the human neural tube. In some embodiments, producing the first chemical gradient along the length of the channel allows for the generation of forebrain-like, midbrain-like, hindbrain-like, and spinal cord-like tissues within the neural tube-like structure. In some embodiments, producing the first chemical gradient along the length of the channel allows for generation of forebrain spheroids, midbrain spheroids, hindbrain spheroids, and/or spinal cord spheroids. Producing a first chemical gradient along the length of the central channel may be performed by applying various compositions to the left and/or right reservoirs of the central channel. In some embodiments, producing a first chemical gradient along the length of the central channel comprises adding a first composition to the left reservoir of the central channel and adding a second composition to the right reservoir of the central channel. In some embodiments, the second composition (e.g. the composition added to the right reservoir of the central channel) promotes differentiation of cells that differ in expression of one or more markers compared to the cells proximal to the left reservoir. Accordingly, in some embodiments the first and second compositions promote generation of different cells along the length of the neural tube-like structure in the central channel, thus enabling selection of first and second compositions that will facilitate the generation of anterior-posterior patterns mimicking those seen in the human neural tube. As another example, in some embodiments the first and second compositions promote generation of different cells in different patterned cell colonies along the length of the central channel, thus enabling selection of first and second compositions that will facilitate the generation of forebrain spheroids, midbrain spheroids, hindbrain spheroids, and/or spinal cord spheroids in the central channel.

In some embodiments, the first composition added to the left reservoir of the central channel contains chemicals that promote the development of cells expressing markers seen in the anterior section of the human neural tube. The second composition added to the right reservoir of the central channel may contain chemicals that promote the development of cells expressing markers seen in the posterior section of the human neural tube. In some embodiments, the first composition added to the left reservoir of the central channel comprises a suitable cell culture medium (e.g. basal medium) supplemented with one or more chemicals to induce neural differentiation. For example, the first composition added to the left reservoir of the central channel may comprise cell culture medium and dual SMAD inhibitors (DSi) (e.g., TGF-β inhibitor (e.g. SB431542), and BMP4 inhibitor (e.g. LDN193189)). The second composition added to the right reservoir of the central channel may comprise the same components present in the first composition (e.g. cell culture medium and DSi), in addition to CHIR, FGF8, and/or RA. In some embodiments, the second composition comprises cell culture medium, DSi, CHIR, and FGF8. In some embodiments, the second composition comprises cell culture medium, DSi, CHIR, FGF8, and RA.

In some embodiments, the methods further comprise generating an orthogonal chemical gradient along the radius of the central channel. The orthogonal chemical gradient may be generated by applying a third composition to the top channel and a fourth composition to the bottom channel. The third composition may be applied to the left and right reservoirs of the top channel to promote an even distribution of the chemicals therein along the length of the top channel (e.g., along the semi-permeable structure separating the top channel from the central channel). The fourth composition may be applied to the left and right reservoirs of the bottom channel to promote an even distribution of the chemicals therein along the length of the bottom channel (e.g. along the semi-permeable structure separating the bottom channel from the central channel).

The neural tube patterns along the dorsal-ventral axis to establish compartments of neural progenitor cells that give rise to distinct classes of neurons. In some embodiments, the third composition and the fourth composition contain chemicals that promote the development of cells expressing markers seen in the dorsal and ventral portions of the human neural tube, respectively. For example, the third composition may contain chemicals that promote the development of cells expressing markers seen in the dorsal portion of the human neural tube and the fourth composition may contain chemicals that promote the development of cells expressing markers seen in the ventral portion of the human neural tube. Bone morphogenic proteins (BMPs) and WNT family members are thought to play an important role in patterning the dorsal axis. Accordingly, the third composition may comprise a bone morphogenic protein or an agonist thereof. For example, the third composition may comprise BMP2, BMP3b, BMP4, BMP5, BMP6, BMP7, BMP9, BMP11, BMP12, BMP13, BMP14, or an agonist thereof, each of which are implicated in the development and patterning of the dorsal neural tube. In some embodiments, the third compositions comprises BMP2 and/or BMP4. For example, the third composition may comprise BMP4. In some embodiments, the third composition comprises WNT-1, WNT-3, WNT-3a, or an agonist thereof.

In some embodiments, the fourth composition contains chemicals that promote the development of cells expressing markers seen in the ventral portion of the human neural tube. For example, sonic hedgehog protein is known to play a role in ventral patterning of the neural tube. Activation of the SHH pathway leads to a disinhibition of smoothened protein (Smo). Accordingly, the fourth composition may contain sonic hedgehog protein (SHH), smoothened protein, or an agonist thereof. For example, the fourth composition may comprise smoothened agonist (SAG). In some embodiments, the fourth composition may comprise SAG and retinoic acid.

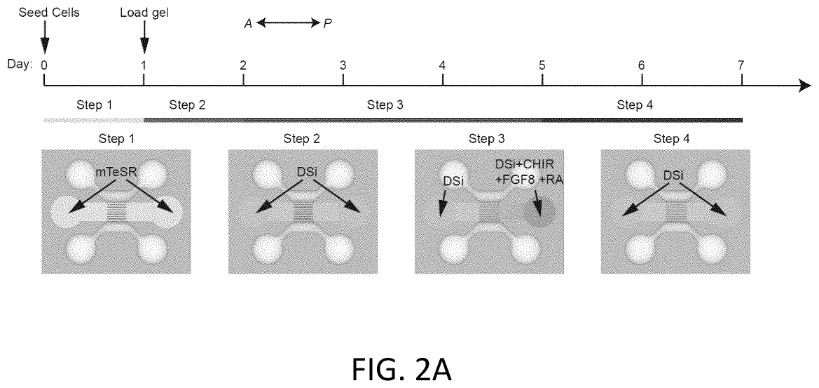

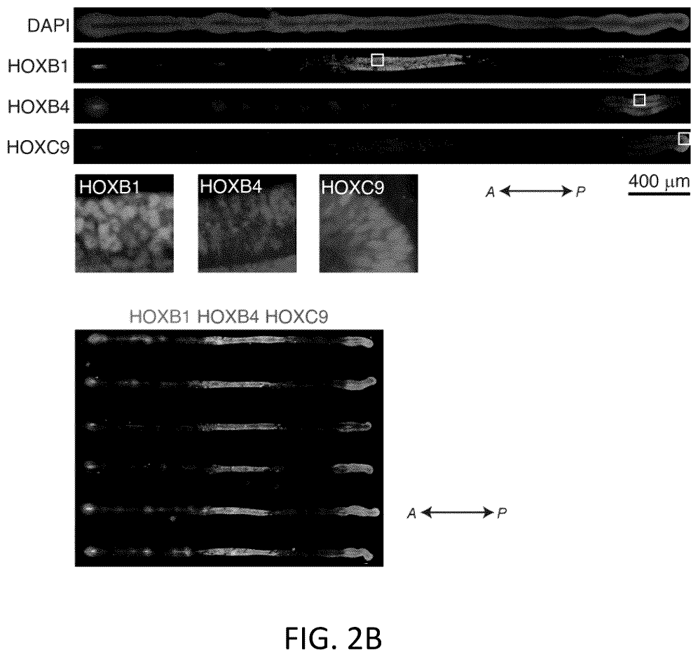

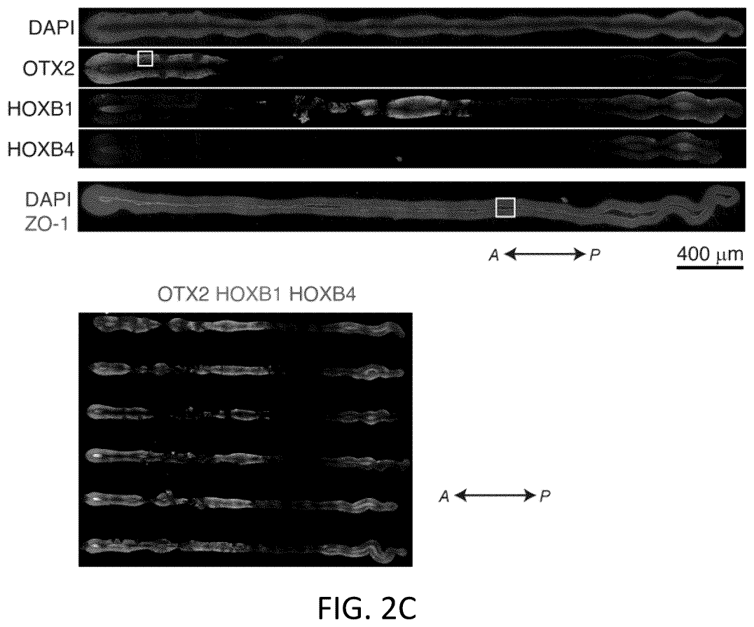

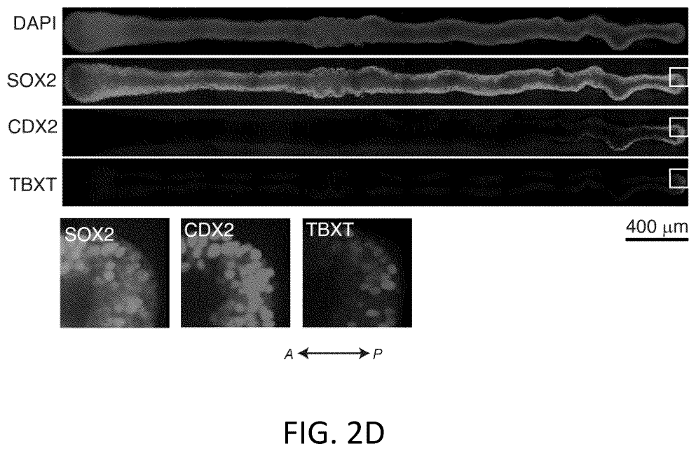

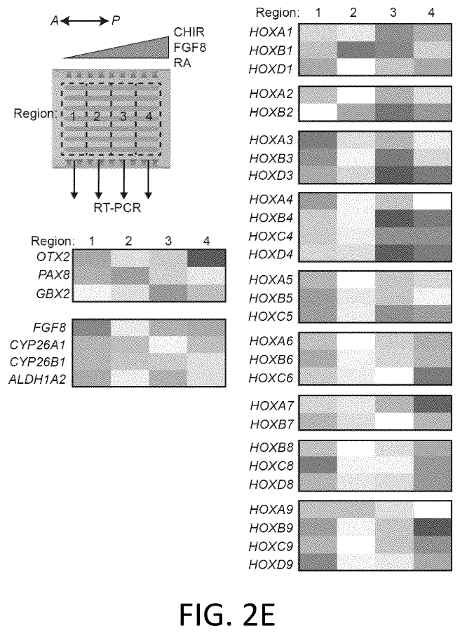

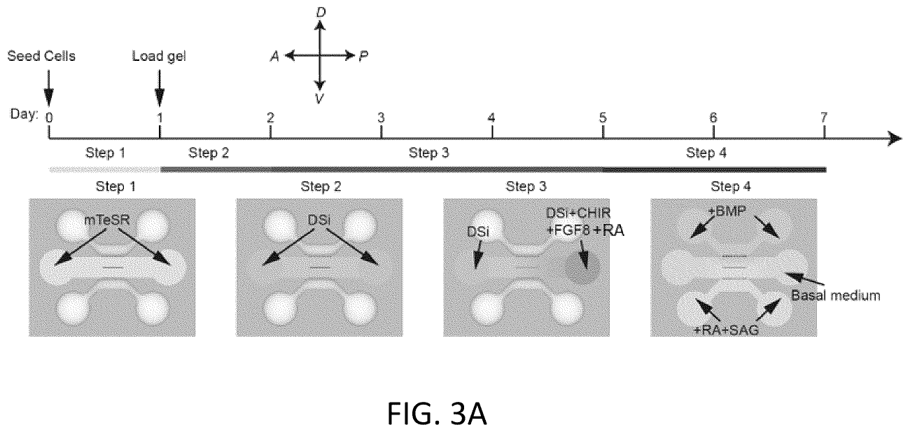

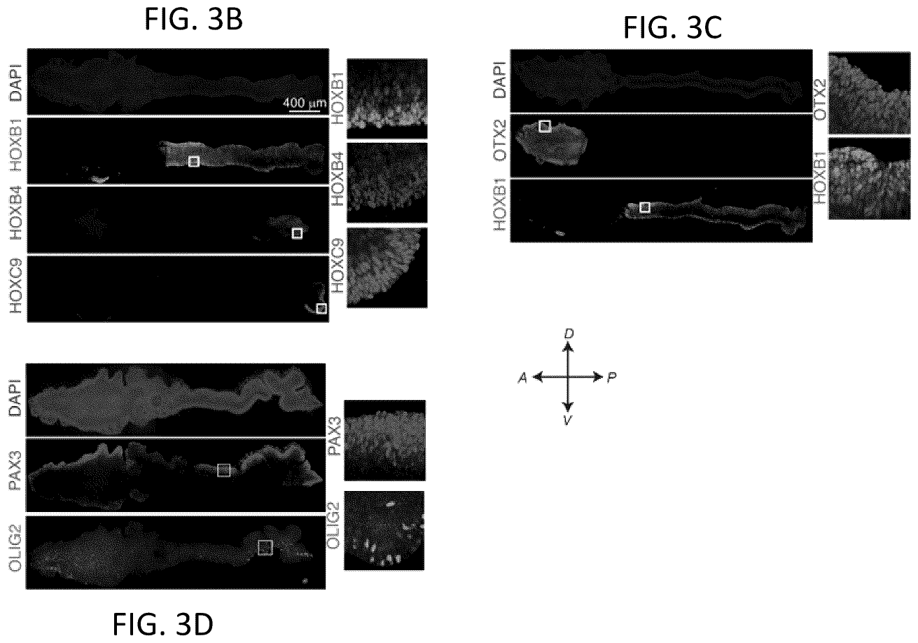

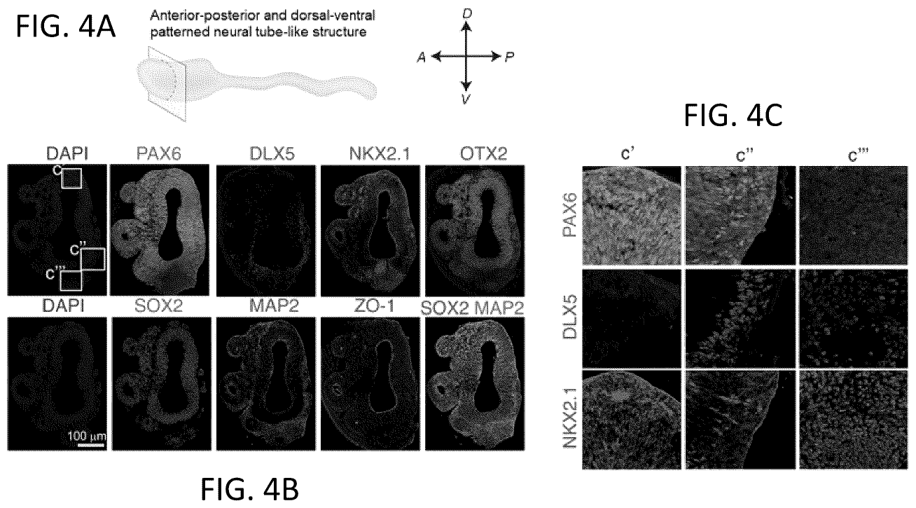

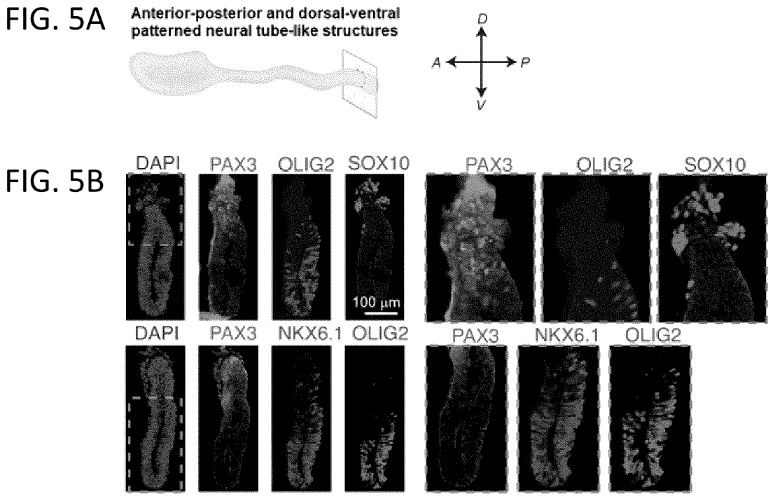

The methods described herein may be employed to facilitate the development of neural tube-like tissues exhibiting anterior-posterior and dorsal-ventral patterning typically seen in the human neural tube. Accordingly, cells in the anterior section, posterior section, dorsal section, and ventral section of the neural tube-like tissue may express markers typically seen in the corresponding section within the human neural tube. Exemplary dorsal-ventral and anterior-posterior patterning is shown for example in FIG. 2B-2D, FIG. 3B-D, FIG. 4 and FIG. 5. In FIG. 5, dorsal neural tube markers PAX3 and SOX10 are expressed on the dorsal side of the neural tube-like tissues, while ventral neural tube markers OLIG2 and NKX6.1 are expressed on the opposite (e.g. ventral) side of the neural tube-like tissue.

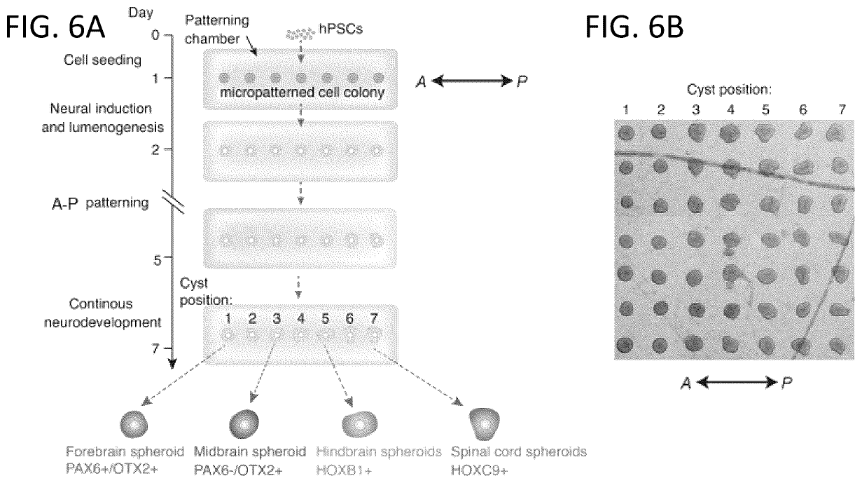

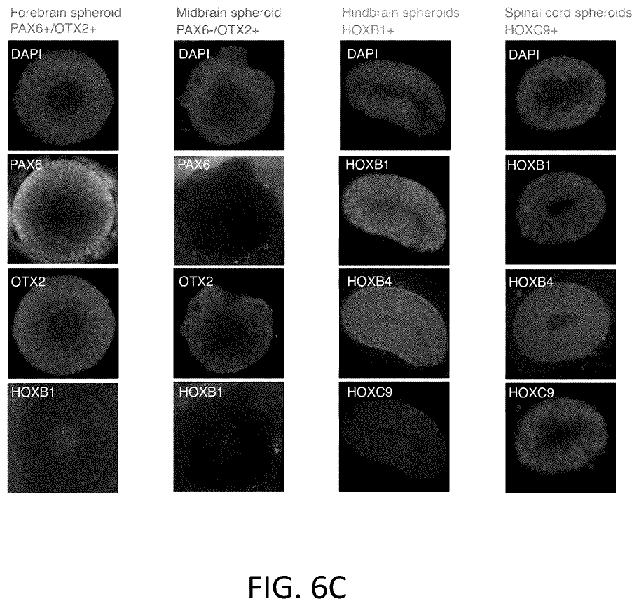

In some embodiments, the methods described herein are used to facilitate the development of neural spheroids. In some embodiments, the first composition added to the left reservoir of the central channel comprises a suitable cell culture medium (e.g. basal medium) supplemented with one or more chemicals to induce neural differentiation. For example, the first composition added to the left reservoir of the central channel may comprise cell culture medium and dual SMAD inhibitors (DSi) (e.g., TGF-β inhibitor (e.g. SB431542), and BMP4 inhibitor (e.g. LDN193189)). The second composition added to the right reservoir of the central channel may comprise the same components present in the first composition (e.g. cell culture medium and DSi), in addition to CHIR, FGF8, and/or RA. In some embodiments, the second composition comprises cell culture medium, DSi, CHIR, FGF8, and RA, which induces posteriorization within the central channel, thereby inducing the formation of forebrain spheroids, midbrain spheroids, hindbrain spheroids, and/or spinal cord spheroids at varying positions along the length of the channel. This concept is shown in FIG. 6A-6B. As shown, cyst position 1 (e.g. a position that in this instance would form a forebrain spheroid) is closest to the left reservoir of the central channel, whereas cyst position 7 (e.g. a position that in this instance would form a spinal cord spheroid) is closest to the right reservoir of the central channel.

In some embodiments the methods described herein are used to facilitate the development of forebrain spheroids, midbrain spheroids, hindbrain spheroids, and/or spinal cord spheroids exhibiting cell types typically seen in the corresponding section of tissue of the neural tube (e.g. the forebrain tissue, the midbrain tissue, the hindbrain tissue, or the spinal cord tissue portion of the neural tube, respectively). For example, the methods may be used to facilitate the development of forebrain spheroids exhibiting cell types typically seen in the forebrain tissues of the neural tube. As another example, the methods described herein may be used to facilitate the development of midbrain spheroids exhibiting cell types typically seen in the midbrain tissues of the neural tube. As another example, the methods described herein may be used to facilitate the development of hindbrain spheroids exhibiting cell types typically seen in the hindbrain tissues of the neural tube. As another example, the methods described herein may be used to facilitate the development of spinal cord spheroids exhibiting cell types typically seen in the spinal cord tissues of the neural tube. Suitable markers for these sections within the neural tube are exemplified in FIG. 6C. PAX6 may be used as a forebrain marker. OTX2 may be used as a forebrain and midbrain marker. HOXB1 may be used as a hindbrain marker. HOXB4 and HOXC9 may be used as spinal cord markers.

As another example, in some embodiments the methods described herein are used to facilitate the development of forebrain spheroids, midbrain spheroids, hindbrain spheroids, and/or spinal cord spheroids exhibiting patterning typically seen in the corresponding section of tissue in the neural tube (e.g. the forebrain tissue, the midbrain tissue, the hindbrain tissue, or the spinal cord tissue portion of the neural tube, respectively). For example, the methods may be used to facilitate the development of forebrain spheroids exhibiting dorsal-ventral patterning typically seen in the forebrain tissues of the neural tube. As another example, the methods described herein may be used to facilitate the development of midbrain spheroids exhibiting dorsal-ventral patterning typically seen in the midbrain tissues of the neural tube. As another example, the methods described herein may be used to facilitate the development of hindbrain spheroids exhibiting dorsal-ventral patterning typically seen in the hindbrain tissues of the neural tube. As another example, the methods described herein may be used to facilitate the development of spinal cord spheroids exhibiting dorsal-ventral patterning typically seen in the spinal cord tissues of the neural tube.

### III. Uses

The tissues (e.g. neural tube-like tissues and/or neural spheroids) described herein find use in a variety of research, diagnostic, and therapeutic applications.

In some embodiments, neural tube-like tissues and/or neural spheroids are utilized in research applications (e.g., study of normal or abnormal brain development and spinal cord development). In some embodiments, tissues are used in gene expression analysis to identify genes or signaling molecules involved in brain development and spinal cord development. In some embodiments, tissues are used to identify abnormal processes resulting in defects in spinal cord and brain development (e.g., defects that may be associated with one or more neural tube defects, such as spina bifida, Chiari malformation, anencephaly and lissencephaly). In some embodiments, tissues may be used to assess potential therapeutic treatments to prevent, improve, or reverse defects in spinal cord and brain development. For example, tissues may be used to investigate potential therapeutic options to prevent in neural tube defects and brain malformation, including defects that may be associated with one or more neural tube defects, such as spina bifida, Chiari malformation, anencephaly, and lissencephaly. As another example, one or more defects during neural tube development may be induced (e.g. by applying varying chemical gradients, toxins, etc. to the device described herein) and various therapeutic options to reverse or improve the defect may be tested.

In some embodiments, the tissues are used for disease modeling and drug development. In some embodiments, neural tube-like tissues, forebrain spheroids, midbrain spheroids, hindbrain spheroids, spinal cord spheroids, or cells obtained using the devices and methods described herein may be derived from stem cells that have been generated from patients. In some embodiments, test compounds may be contacted with the neural tube-like tissues, forebrain spheroid, midbrain spheroid, hindbrain spheroid, and/or spinal cord spheroid to determine the effect of the compound. In some embodiments, tissues or cells obtained using the devices and methods described herein may be used in drug testing or drug toxicity screening applications. For example, in some embodiments, drugs or biological or environmental agents are tested. Indications for drug testing include any compound or biological agent in the pharmaceutical discovery and development stages, or drugs approved by drug regulatory agencies, like the US Federal Drug Agency. All classes of drugs, over-the-counter and nutraceuticals for any medical indications are known or suspected environmental toxicant may be utilized. In some embodiments, drugs are screened using the tissues described herein to identify drugs that are potentially toxic to the developing spinal cord or brain regions, such as toxic during neurodevelopment in utero. Alternatively, drugs may be screened using the tissues described herein for general toxicity. In some embodiments, screening methods are high throughput screening methods.

Embodiments of the present disclosure provide kits comprising the tissues or cells described herein. For example, in some embodiments, kits comprise tissues (e.g., neural tube-like tissues, forebrain spheroids, midbrain spheroids, hindbrain spheroids, spinal cord spheroids) or stem cells for use in generating the tissues described herein. The kit may further comprise one or more devices as described herein. In some embodiments, kits further comprise reagents for differentiation or use of the stem cells or tissues described herein (e.g., buffers, culture mediums, chemical gradients, test compounds, controls, etc.).

### EXAMPLES

The following examples are provided in order to demonstrate and further illustrate certain preferred embodiments and aspects of the present disclosure and are not to be construed as limiting the scope thereof.

### Example 1

### Device Fabrication

The microfluidic device consists of a polydimethylsiloxane (PDMS) structure layer attached onto a coverslip. Adhesive islands of different shapes (rectangular shape or circular shape) were first printed onto the coverslip using microcontact printing before the assembly of the PDMS structure layer and coverslip.

The PDMS structure layer was made by mixing PDMS curing agent and base polymer (Sylgard 184; Dow Corning) before casting PDMS prepolymer onto a microfabricated silicon mold and baking at 110° C. for 1 hour. Medium reservoirs (6 mm in diameter) and loading ports (1 mm in diameter) were then punched into the PDMS structure layer using Harris Uni-Core punch tools (Ted Pella).

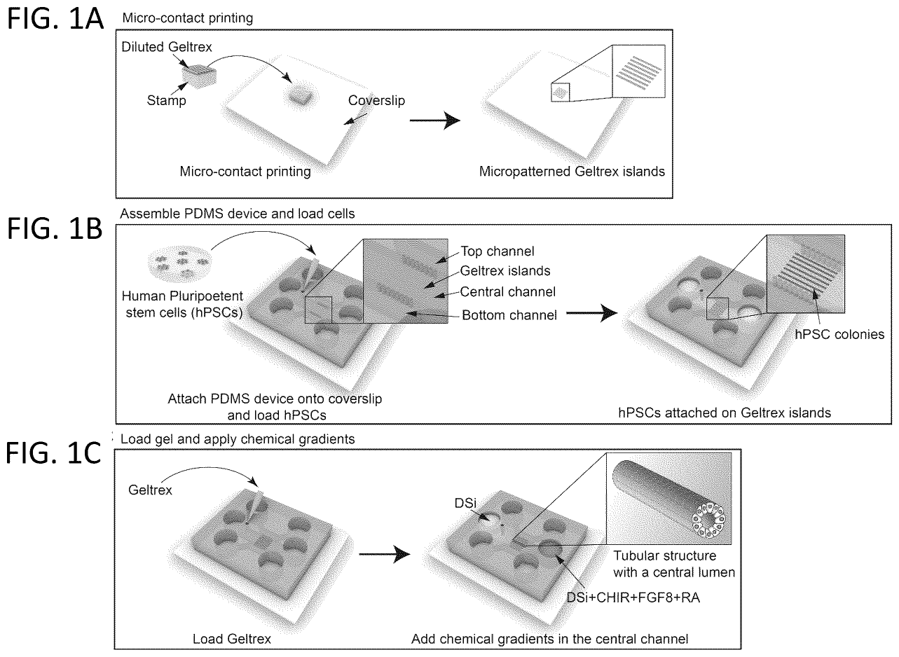

In parallel, PDMS stamps containing different micropatterns (rectangular shape or circular shape) were fabricated by casting PDMS prepolymer onto a microfabricated silicon mold and baking at 110° C. for 1 hour. Then PDMS stamps were immersed in a 1% Geltrex solution (v/v) at 4° C. overnight. The next day, coverslips were sonicated in 100% ethanol for 30 min and treated with ultraviolet zone for 7 min. Then PDMS stamps coated with Geltrex were blown dry under nitrogen and placed in conformal contact with ultraviolet ozone-treated coverslips to transfer the Geltrex adhesive patterns onto coverslips (FIG. 1A). PDMS structure layers were then attached onto coverslips with the microcontact printed Geltrex islands in the center of central channel (FIG. 1B).

### Microfluidic Assay

Colonies of human pluripotent stem cells (hPSCs) in tissue culture plates were dissociated by Accutase (Sigma-Aldrich) at 37° C. for 8 min before being suspended in mTeSR medium (STEMCELL Technologies) as single cells. Cells were then centrifuged and re-suspended in mTeSR containing 10 μM Y27632 (Tocris), a ROCK inhibitor that prevents dissociation-induced apoptosis of hPSCs, at a concentration of 9×10⁶ cells per ml. 10 μl hPSC suspension was introduced into the central channel. Medium reservoirs were filled with fresh mTeSR medium containing 10 μM Y27632 (Day 0) (FIG. 1B). On day 1, 100% Geltrex was introduced into the central channel after aspirating mTeSR medium to establish a 3D culture environment in the central channel. Medium in the left and right reservoirs of the central channel was switched to a fresh basal medium supplemented with dual SMAD inhibitors (DSi; TGF-β inhibitor SB431542, 10 μM, STEMCELL Technologies; BMP4 inhibitor LDN193189, 200 nM, STEMCELL Technologies) to induce neural differentiation (FIG. 1C). Basal medium comprised 1:1 mixture of DMEM/F12 and neurobasal medium (GIBCO), 1% N2 supplement (GIBCO), 2% B-27 supplement (GIBCO), 2 mM glutamax and 1% non-essential amino acids.

To generate anterior-posterior patterned neural tube-like tissues, CHIR99021 (CHIR, WNT activator, 3 μM), FGF8 (200 ng/ml) and/or RA (500 nM) were supplemented into the right medium reservoir of the central channel in addition to DSi from day 2 to day 5.

Medium was switched to basal medium supplemented with DSi from day 5 to day 7 (FIG. 2A & FIG. 3A).

To generate anterior-posterior and dorsal-ventral patterned neural tube-like tissues, CHIR99021 (CHIR, WNT activator, 3 μM), FGF8 (200 ng/ml) and/or retinoic acid (RA, 500 nM) were supplemented into the right medium reservoir of the central channel in addition to DSi from day 2 to day 5. From day 5 to day 7, the top channel was then filled with the basal medium supplemented with BMP4 (50 ng/ml), while the bottom channel was filled with the basal medium supplemented with RA (500 nM) and smoothened agonist (SAG, 500 nM) (FIG. 3A).

To generate forebrain-like, midbrain-like, hindbrain-like, and spinal cord-like tissues, arrays of circular adhesive islands were first printed onto the coverslip using microcontact printing before the assembly of the PDMS structure layer and coverslip. From day 2 to day 5, CHIR (3 μM), FGF8 (200 ng/ml) and/or RA (500 nM) were supplemented into the right medium reservoir of the central channel in addition to DSi. Medium was switched to basal medium supplemented with DSi from day 5 to day 7 (FIG. 6A).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method comprising:
a) providing a microfluidic device comprising:
i. a central channel comprising a plurality of cell-attachment islands positioned on a bottom surface of the central channel;
ii. a top channel parallel to the central channel, wherein the top channel is separated from the central channel by a first semi-permeable structure; and
iii. a bottom channel parallel to the central channel, wherein the bottom channel is separated from the central channel by a second semi-permeable structure;
wherein said position of the top, central and bottom channels refers to the position of said channels in an upper view of the microfluidic device;
b) introducing stem cells into the central channel and promoting attachment of the stem cells to the cell-attachment islands;
c) injecting a gel matrix into the central channel and promoting formation of a three-dimensional tubular structure;
d) producing a first chemical gradient along the length of the central channel; and
e) producing an orthogonal chemical gradient along the radius axis of the central channel, wherein production of the first chemical gradient and the orthogonal chemical gradient promotes generation of a neural tube-like tissue exhibiting anterior-posterior and dorsal-ventral patterns mimicking those seen in the human neural tube, or promotes generation of at least one neural spheroid.

2. The method of claim 1, wherein the at least one neural spheroid comprises a forebrain spheroid, a midbrain spheroid, a hindbrain spheroid, and/or a spinal cord spheroid.

3. The method of claim 1, wherein the first chemical gradient and the orthogonal chemical gradient are produced simultaneously.

4. The method of claim 1, wherein the stem cells comprise human pluripotent stem cells (hPSCs).

5. The method of claim 1, wherein promoting attachment to the cell-attachment islands comprises incubating the stem cells within the central channel for 16 hours at 37° C.

6. The method of claim 1, wherein promoting formation of a three-dimensional cellular structure comprises incubating the stem cells and the gel matrix within the central channel for 24 hours at 37° C.

7. The method of claim 1, wherein:
a) the first chemical gradient is produced by applying a first composition to the left reservoir of the central channel and applying a second composition to the right reservoir of the central channel; and
b) the orthogonal chemical gradient is produced by applying a third composition to the top channel and applying a fourth composition to the bottom channel.

8. The method of claim 7, wherein the third composition is applied to the left and right reservoirs of the top channel and the fourth composition is applied to the left and right reservoirs of the bottom channel.

9. The method of claim 1, wherein the central channel comprises at least one reservoir in fluid connection with the central channel.

10. The method of claim 9, wherein the central channel comprises a right reservoir and a left reservoir, wherein each reservoir is in fluid connection with the central channel.

11. The method of claim 1, wherein the top channel comprises at least one reservoir in fluid connection with the top channel.

12. The method of claim 11, wherein the top channel comprises a right reservoir and a left reservoir, wherein each reservoir is in fluid connection with the top channel.

13. The method of claim 1, wherein the bottom channel comprises at least one reservoir in fluid connection with the bottom channel.

14. The method of claim 13, wherein the bottom channel comprises a right reservoir and a left reservoir, wherein each reservoir is in fluid connection with the bottom channel.

15. The method of claim 1, wherein the first semi-permeable structure and the second semi-permeable structure comprise a plurality of circular microposts.

16. The method of claim 1, wherein the cell-attachment islands are rectangular in shape or circular in shape, and/or wherein the cell-attachment islands comprise a diluted gel matrix.

17. The method of claim 1, wherein the microfluidic device comprises at least 4 cell-attachment islands.

18. The method of claim 1, wherein the cell-attachment islands are aligned parallel to each other on the bottom surface of the central channel, or wherein the cell-attachment islands are arranged in a regular array format on the bottom surface of the central channel.

19. The method of claim 1, further comprising contacting the neural-tube like tissue or the neural spheroid with a test compound, and determining an effect of the test compound on the neural-tube like tissue or neural spheroid.
